(19) United States
(12) Patent Application Publication
(10) Pub. No.: US 2024/0066271 A1
(43) Pub. Date: Feb. 29, 2024

(54) Integrated Catheter System with Stabilization

(71) Applicant: Becton, Dickinson and Company, Franklin Lakes, NJ (US)

(72) Inventors: Jonathan Karl Burkholz, Salt Lake City, UT (US); Serena Robinson Agrawal, Salt Lake City, UT (US); Jason Andrew Wine, Brea, CA (US); Dorien Gemma Butter, Millcreek, UT (US)

(21) Appl. No.: 18/238,597

(22) Filed: Aug. 28, 2023

Related U.S. Application Data
(60) Provisional application No. 63/401,913, filed on Aug. 29, 2022.

(51) Int. Cl. A61M 25/06; A61M 25/00; A61M 25/02; A61M 39/10

(52) U.S. Cl. CPC A61M 25/0606; A61M 25/0097; A61M 25/02; A61M 39/10; A61M 2025/0253; A61M 2039/1016

(57) ABSTRACT

An integrated catheter system includes a catheter adapter having a catheter, a body receiving the catheter, and a side port in fluid communication with the catheter, an integrated extension set including a proximal access port coupled to a proximal end portion of the integrated extension set, with the integrated extension set in fluid communication with the side port of the catheter adapter, and a stabilization platform spaced from the body of the catheter adapter and positioned along the integrated extension set.

### CROSS-REFERENCE TO RELATED APPLICATION

The present application claims priority to U.S. Provisional Application No. 63/401,913 entitled “Integrated Catheter System with Stabilization” filed Aug. 29, 2022, the entire disclosure of which is hereby incorporated by reference in its entirety.

### BACKGROUND OF THE INVENTION

### Field of the Invention

The present disclosure relates to an integrated catheter system with a stabilization platform.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (“PIVC”). The over-the-needle catheter may be mounted over an introducer needle having a sharp distal tip. The catheter and the introducer needle may be assembled so that the distal tip of the introducer needle extends beyond the distal tip of the catheter with the bevel of the needle facing up away from a skin surface of the patient. The catheter and introducer needle are generally inserted at a shallow angle through the skin into the vasculature of the patient. In order to verify the proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Blood withdrawal using a peripheral IV catheter may be difficult for several reasons, particularly when the indwelling time of the catheter is more than one day. For example, when the catheter is left inserted in the patient for a prolonged period of time, the catheter or vein may be more susceptible to narrowing, collapse, kinking, blockage by debris (e.g., fibrin or platelet clots), and adhering of a tip of the catheter to the vasculature. Due to this, catheters may often be used for acquiring a blood sample at the time of catheter placement but are much less frequently used for acquiring a blood sample during the catheter dwell period.

Accordingly, blood draw devices have been developed to collect blood samples through an existing PIVC. Blood draw devices attach to the PIVC and include a flexible flow tube that is advanced through the PIVC, beyond the catheter tip, and into a vessel to collect a blood sample. After blood collection, the blood draw device is removed from the PIVC and discarded. One example of a blood draw device is shown and described in U.S. Pat. No. 11,090,461, which is hereby incorporated by reference in its entirety.

### SUMMARY OF THE INVENTION

In one aspect or embodiment, an integrated catheter system includes a catheter adapter having a catheter, a body receiving the catheter, and a side port in fluid communication with the catheter, an integrated extension set comprising a proximal access port coupled to a proximal end portion of the integrated extension set, with the integrated extension set in fluid communication with the side port of the catheter adapter, and a stabilization platform spaced from the body of the catheter adapter and positioned along the integrated extension set.

The system may include a near patient access port in fluid communication with the side port of the catheter adapter, with the near patient access port including a connector portion configured to be coupled to a peripheral probe device. The near patient access port may further include a secondary port, with the secondary port coupled to the integrated extension set. The connector portion of the near patient access port may be a needle-free connector.

The stabilization platform may include an attachment member configured to secure the stabilization platform to the skin surface of a patient. The attachment member may include an adhesive pad.

The system may include a clamp positioned along the integrated extension set, with the stabilization platform is positioned distal to the proximal access port and proximal to the clamp. The system may include a clamp positioned along the integrated extension set, with the stabilization platform is positioned distal to the clamp. The stabilization platform may be integrated or coupled to the proximal access port. The stabilization platform may be integrated or coupled to the near patient access port. The stabilization platform may include a pair of stabilizing wings. The catheter adapter may include a pair of catheter adapter stabilizing wings. The stabilization platform may be moveable along the integrated extension set.

The system may include a force controlled release connector coupled to the proximal access port, with at least a portion of the force controlled release connector configured to separate from the proximal access port when a predetermined separation force is applied to the force controlled release connector. The proximal access port may include a needle-free connector. The force controlled release connector may include a disconnect connector and a release feature coupled to the needle-free connector of the proximal access port, with the release feature engaged with the disconnect connector and configured to be disengaged from the disconnect connector when a predetermined separation force is applied to the disconnect connector or the needle-free connector of the proximal access port. The disconnect connector may include a valve member. The disconnect connector may include a first line extending from the disconnect connector and a second line extending from the disconnect connector, with the first line including a first port and the second line including a second port.

The force controlled release connector may include a first connector in fluid communication with the catheter adapter and a second connector connected to the first connector, with the second connector coupled to and in fluid communication with the integrated extension set. The system may include a secondary stabilization platform configured to stabilize the catheter adapter and the force controlled release connector, with the first connector of the force controlled release connector coupled to the catheter adapter via connection tubing. The second connector may be configured to be connected to a peripheral probe device or a needle insertion assembly.

### BRIEF DESCRIPTION OF THE DRAWINGS

FIG. 1 is a top view of an integrated catheter system according to one aspect or embodiment of the present application;

FIG. 2 is a perspective view of an integrated catheter system according to a further aspect or embodiment of the present application;

FIG. 3 is a perspective view of an integrated catheter system according to a further aspect or embodiment of the present application;

FIG. 4 is a perspective view of an integrated catheter system according to a further aspect or embodiment of the present application;

FIG. 5 is a top view of an integrated catheter system according to a further aspect or embodiment of the present application;

FIG. 6 is a top view of an integrated catheter system according to a further aspect or embodiment of the present application;

FIG. 7 is a top view of a force controlled release connection according to one aspect or embodiment of the present application;

FIG. 8 is a cross-sectional view of a force controlled release connection according to a further aspect or embodiment of the present application;

FIG. 9 is a perspective view of an integrated catheter system according to a further aspect or embodiment of the present application;

FIG. 10 is a schematic view of an integrated catheter system according to a further aspect or embodiment of the present application, showing the system during catheter placement; and

FIG. 11 is a schematic view of the integrated catheter system of FIG. 10, showing the system during catheter indwell.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a catheter insertion device in preparation for or during use. Similarly, in this application, the terms “in the distal direction” and “distally” mean in the direction toward the distal tip of the needle or catheter of the system, and the terms “in the proximal direction” and “proximally” mean in the direction opposite the direction of the distal tip of the needle or catheter.

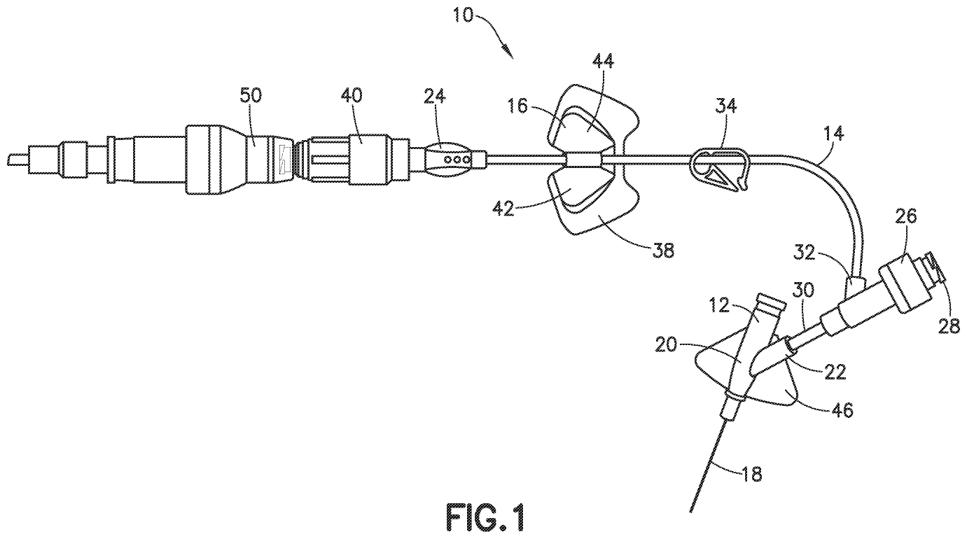

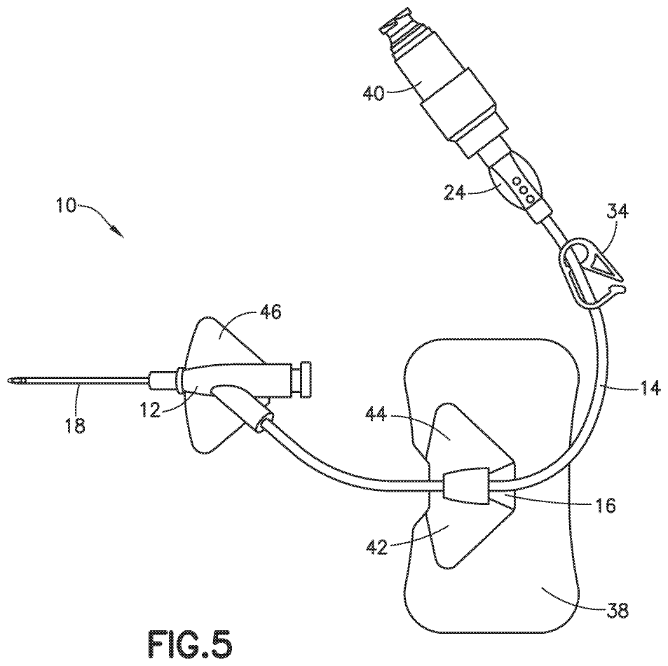

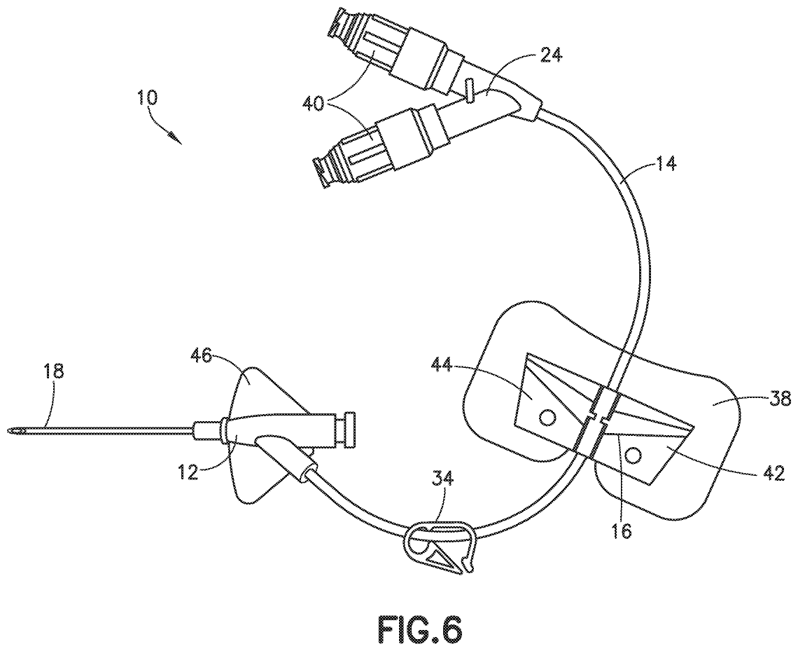

Referring to FIGS. 1-6, according to one aspect or embodiment, an integrated catheter system 10 includes a catheter adapter 12, an integrated extension set 14, and a stabilization platform 16. The catheter adapter 12 includes a catheter 18, a body 20 receiving the catheter 18, and a side port 22 in fluid communication with the catheter 18. The integrated extension set 14 includes a proximal access port 24 coupled to a proximal end portion of the integrated extension set 14, with the integrated extension set 14 in fluid communication with the side port 22 of the catheter adapter 12. The stabilization platform 16 is spaced from the body 20 of the catheter adapter 12 and positioned along the integrated extension set 14. The stabilization platform 16 is configured to provide a primary securement point to a skin surface of a patient and minimize catheter dislodgement, infiltration, or catheter movement that may result in a tip of the catheter 18 from being pulled out of a vein or artery. The stabilization platform 16 is configured to isolate external applied forces from the catheter adapter 12 and insertion site to limit movement of the catheter 18 and/or dislodgement of the catheter 18 due to forces being applied to an intravenous line or extension sets. The system 10 is configured to provide sufficient compliance, flex, or slack between the stabilization platform 16 and the catheter adapter 12 to allow some displacement of the stabilization platform 16 relative to the catheter 18 due to movement of the skin around the stabilization platform 16 without applying excessive force and displacement of the catheter adapter 12 itself. In some aspects or embodiments, the system 10 provides a high pressure rated catheter with blood draw and instrument delivery access and high pressure injection capabilities.

Referring to FIG. 1, for example, the system 10 may include a near patient access port 26 in fluid communication with the side port 22 of the catheter adapter 12, with the near patient access port 26 including a connector portion 28 configured to be coupled to a peripheral probe device. The near patient access port 26 is configured to provide catheter access to peripheral devices such as, e.g., a blood draw device (e.g., PIVO™ from Becton, Dickinson and Company), or a vascular access probe (VAP) for in-vein digital measurement of patient data such as temperature, pH, lactate, and/or other blood-based measurements. In some embodiments, the near patient access port 26 is coupled to the side port 22 of the catheter adapter 12 via a length of intermediate tubing 30. However, in other embodiments, the near patient access port 26 may be coupled directly to the side port 22, or connected via another intermediate member.

The near patient access port 26 includes the connector portion 24, which in some embodiments, is configured to be compatible with peripheral devices such as blood draw devices and/or vascular access probes. In some embodiments, the connector portion 28 is configured as a needle-free connector (NFC) configured to receive a blunt introducer of a blood draw device. More specifically, the connector portion 28 may be configured as a split-septum NFC with direct probe access such as, e.g., Q-Syte™ or SmartSite™ NFCs from Becton, Dickinson and Co., or any other appropriate split-septum NFC. Alternatively, in other embodiments, the connector portion 28 may be formed of a non-split-septum-type NFC. Furthermore, in some embodiments, the near patient access port 26 may include anti-microbial and/or flush-promoting features. For example, the near patient access port 26 may include one or more of an offset tubing port vortex-creating feature, a proximal flow-diverting feature, anti-microbial NFC lubricant, anti-microbial eluting surface coating(s) or insert(s), etc. With the near patient access port 26 fluidly coupled to the catheter adapter 12 via the side port 22, the system 10 provides for probe (or tube) access from a peripheral probe device through the indwelling catheter 18. The near patient access port 26 further includes a secondary port 32 positioned near a distal end thereof. In some embodiments, the secondary port 32 is coupled to the integrated extension set 14. A clamp 34 may be provided on the integrated extension set 14, with the clamp 34 configured to selectively restrict flow through the integrated extension set 14.

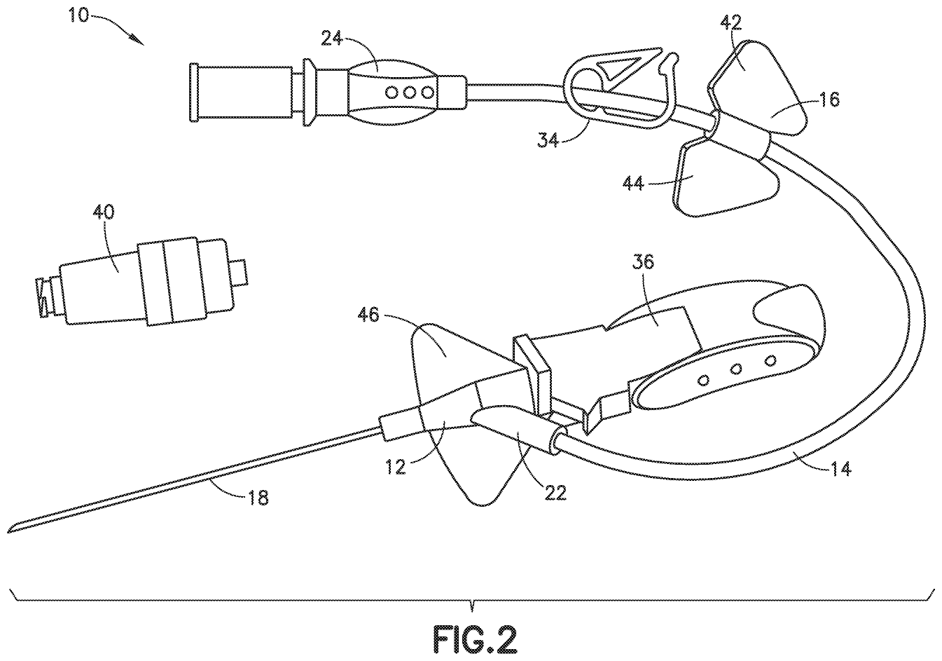

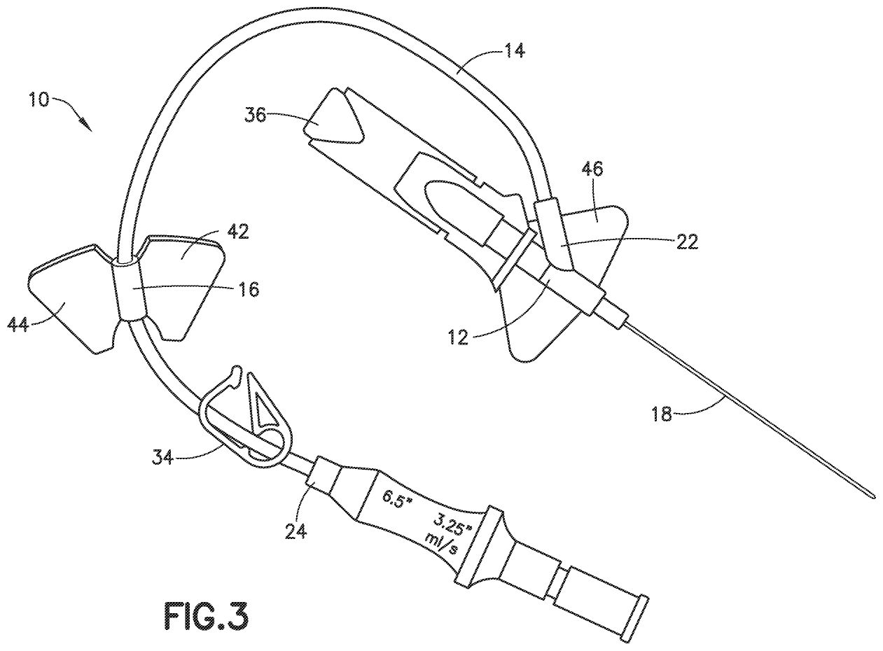

Referring to FIGS. 2 and 3, in some aspects or embodiments, the integrated extension set 14 extends directly from the side port 22 of the catheter, with the system 10 not including the near patient access port 26. The system 10 may incorporate the BD Nexiva™ closed peripheral IV catheter system (FIG. 2) or the BD Nexiva™ Diffusics™ closed IV catheter system (FIG. 3). The catheter 18 of any of the aspects or embodiments described herein may be inserted according to any suitable arrangement or method. The system 10 may include a needle insertion device 36 to facilitate the insertion of the catheter 18.

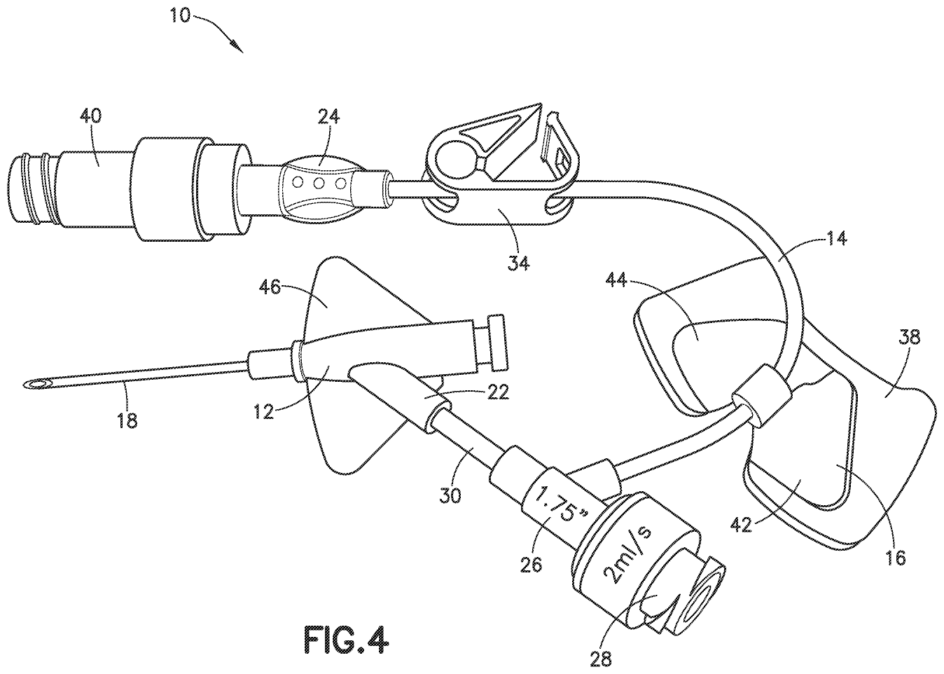

Referring to FIGS. 4-6, in one aspect or embodiment, the stabilization platform 16 includes an attachment member 38 configured to secure the stabilization platform 16 to a skin surface of a patient. The attachment member 38 may include an adhesive pad. The attachment member 38 may be securement tape, an external stabilization device (ESD), dressing, and/or an anchoring platform adhesive with paper backing and skin adhesive. In one aspect or embodiment, the stabilization platform 16 is shaped to fit within the StatLock™ ESD product from Becton, Dickinson and Co.

Referring again to FIGS. 1-6, in some aspects or embodiments, the stabilization platform 16 is positioned distal to the proximal access port 24 and proximal to the clamp 34. In some aspects or embodiments, the stabilization platform 16 is positioned distal to the clamp 34. The stabilization platform 16 may also be integrated or coupled to the proximal access port 24 or the near patient access port 26. The proximal access port 24 may include a needle-free connector 40 integrated into the proximal access port 24 or separately connected to the proximal access port 24. The stabilization platform 16 includes a pair of stabilizing wings 42, 44, although other suitable arrangements may be utilized. The stabilization platform 16 may be fixed, removable, or moveable along the integrated extension set. The stabilization platform 16 may be oval, triangular, round, square, rectangular, trapezoidal, or other suitable shape. The stabilization platform 16 may be flexible, soft, semi-rigid, or rigid and may include features or a living hinge to promote flexibility for patient comfort. The stabilization platform 16 may also include one or more features to improve attachment to the attachment member 38, such as protrusions, surface roughness, pockets, etc. The stabilization platform 16 may include one or more colors or visual marking to provide catheter gauge, length, type, or procedure related information. In some aspects or embodiments, the catheter adapter 12 includes a pair of catheter adapter stabilizing wings 46.

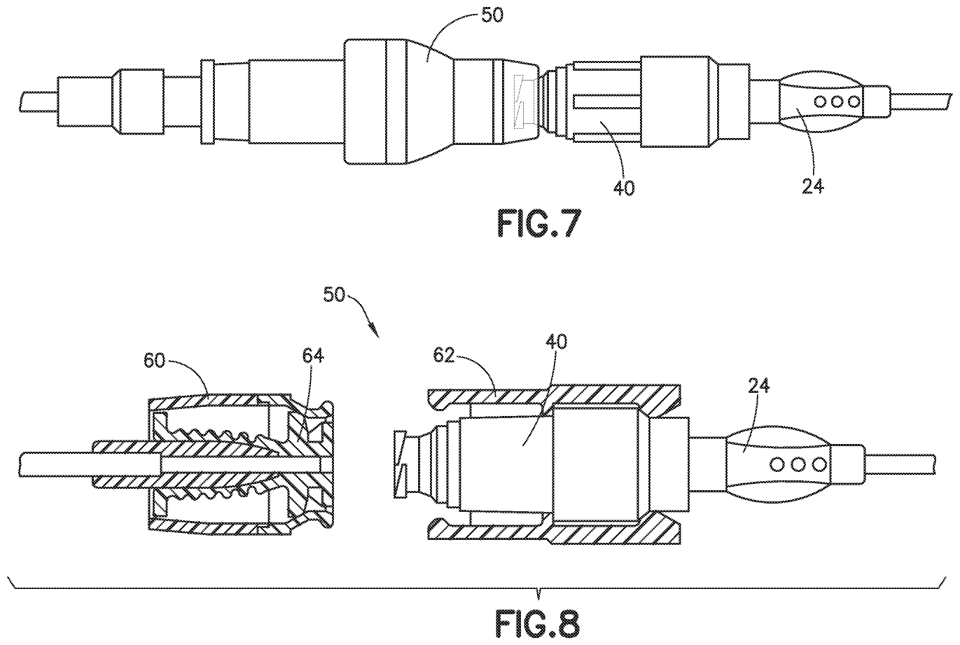

Referring to FIGS. 1 and 7-9, in some aspects or embodiments, the system 10 further includes a force controlled release connector 50 coupled to the proximal access port 24. At least a portion of the force controlled release connector 50 is configured to separate from the proximal access port 24 when a predetermined separation force is applied to the force controlled release connector 50. At least a portion of the force controlled release connector 50 is configured to detach from the integrated extension set 14 to prevent excessive forces from being transmitted to the catheter adapter 12 via the integrated extension set 14. For example, a line from an IV bag may be connected to the integrated extension set 14 via the force controlled release connector 50 such that excessive movement of the IV bag and line will merely detach the connection between the integrated extension set 14 and the force controlled connector 50 rather than disrupting the placement of the catheter 18.

Referring to FIG. 8, the force controlled release connector 50 includes a disconnect connector 60 and a release feature 62 coupled to the needle-free connector 40 of the proximal access port 24. The release feature 62 is engaged with the disconnect connector 60 and configured to be disengaged from the disconnect connector 60 when a predetermined separation force is applied to the disconnect connector 60 or the needle-free connector 40 of the proximal access port 24. The disconnect connector 60 includes a valve member 64, which is configured to seal the disconnect connector 60 when separated from the needle-free connector 40. In one aspect or embodiment, the release feature 62 is configured to be biased radially outward when the predetermined separate force is reached. The release feature 62 and the disconnect connector 60 may form a camming arrangement where an axial force causes the release feature 62 to move radially outward, thereby releasing the disconnect connector 60 from the release feature 62. The release feature 62 may be formed integrally with the needle-free connector 40 or may be a separate component attached or coupled to the needle-free-connector 40 or the proximal access port 24.

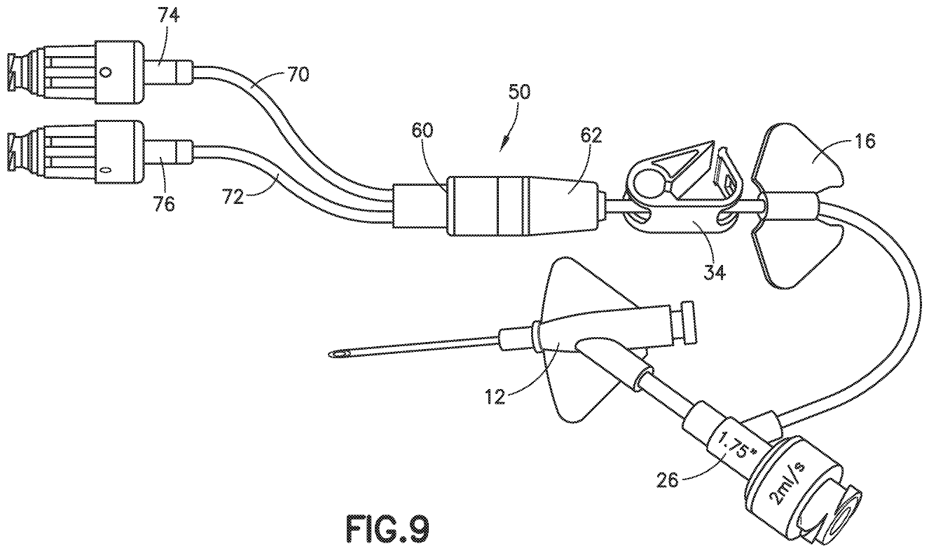

Referring to FIG. 9, in some aspects or embodiments, the disconnect connector 60 includes a first line 70 extending from the disconnect connector and a second line 72 extending from the disconnect connector 60. The first line 70 includes a first port 74 and the second line includes a second port 76. The first and second ports 74, 76 may each include a needle-free connector.

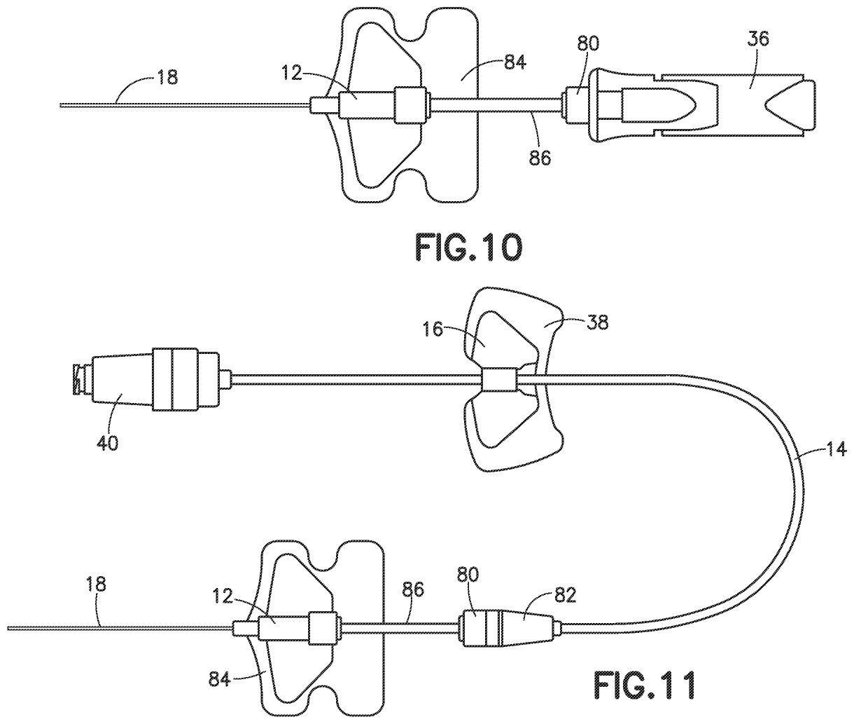

Referring to FIGS. 10 and 11, in a further aspect or embodiment, the force controlled release connector 50 includes a first connector 80 in fluid communication with the catheter adapter 12 and a second connector 82 connected to the first connector 80, with the second connector 82 coupled to and in fluid communication with the integrated extension set 14. A secondary stabilization platform 84 extending between the catheter adapter 12 and the force controlled release connector 50 is configured to stabilize the catheter adapter 12 and the force controlled release connector 50. The first connector 80 of the force controlled release connector 50 is coupled to the catheter adapter via connection tubing 86. The connection tubing 86 is configured to orient the first connector 80 to the direction of tension on the IV line and avoid imparting bending loads on the catheter 18. The second connector 82 is configured to be connected to a peripheral probe device or the needle insertion device 36. The second connector 82 may include a valve member and enable fluid transfer when connected to the first connector 80.

While several embodiments of integrated catheter systems were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

The invention claimed is:

1. An integrated catheter system comprising:
a catheter adapter comprising a catheter, a body receiving the catheter, and a side port in fluid communication with the catheter;
an integrated extension set comprising a proximal access port coupled to a proximal end portion of the integrated extension set, the integrated extension set in fluid communication with the side port of the catheter adapter; and
a stabilization platform spaced from the body of the catheter adapter and positioned along the integrated extension set.

2. The system of claim 1, further comprising a near patient access port in fluid communication with the side port of the catheter adapter, the near patient access port comprising a connector portion configured to be coupled to a peripheral probe device.

3. The system of claim 2, wherein the near patient access port further comprises a secondary port, and wherein the secondary port is coupled to the integrated extension set.

4. The system of claim 2, wherein the connector portion of the near patient access port is a needle-free connector.

5. The system of claim 1, wherein the stabilization platform comprises an attachment member configured to secure the stabilization platform to a skin surface of a patient.

6. The system of claim 5, wherein the attachment member comprises an adhesive pad.

7. The system of claim 1, further comprising a clamp positioned along the integrated extension set, wherein the stabilization platform is positioned distal to the proximal access port and proximal to the clamp.

8. The system of claim 1, further comprising a clamp positioned along the integrated extension set, wherein the stabilization platform is positioned distal to the clamp.

9. The system of claim 1, wherein the stabilization platform is integrated or coupled to the proximal access port.

10. The system of claim 2, wherein the stabilization platform is integrated or coupled to the near patient access port.

11. The system of claim 1, wherein the stabilization platform comprises a pair of stabilizing wings.

12. The system of claim 1, wherein the catheter adapter comprises a pair of catheter adapter stabilizing wings.

13. The system of claim 1, wherein the stabilization platform is moveable along the integrated extension set.

14. The system of claim 1, further comprising a force controlled release connector coupled to the proximal access port, wherein at least a portion of the force controlled release connector is configured to separate from the proximal access port when a predetermined separation force is applied to the force controlled release connector.

15. The system of claim 14, wherein the proximal access port comprises a needle-free connector.

16. The system of claim 15, wherein the force controlled release connector comprises a disconnect connector and a release feature coupled to the needle-free connector of the proximal access port, the release feature is engaged with the disconnect connector and configured to be disengaged from the disconnect connector when a predetermined separation force is applied to the disconnect connector or the needle-free connector of the proximal access port.

17. The system of claim 16, wherein the disconnect connector comprises a valve member.

18. The system of claim 16, wherein the disconnect connector comprises a first line extending from the disconnect connector and a second line extending from the disconnect connector, and wherein the first line comprises a first port and the second line comprises a second port.

19. The system of claim 16, wherein the force controlled release connector comprises a first connector in fluid communication with the catheter adapter and a second connector connected to the first connector, the second connector coupled to and in fluid communication with the integrated extension set.

20. The system of claim 19, further comprising a secondary stabilization platform configured to stabilize the catheter adapter and the force controlled release connector, wherein the first connector of the force controlled release connector is coupled to the catheter adapter via connection tubing.
